(19) United States
(12) Patent Application Publication
(10) Pub. No.: US 2024/0270665 A1
(43) Pub. Date: Aug. 15, 2024

(54) METHOD FOR GENERATING ISOBUTENE, CATALYST FOR GENERATING ISOBUTENE, AND ISOBUTENE GENERATION SYSTEM

(71) Applicants: HONDA MOTOR CO., LTD., Tokyo (JP); NATIONAL INSTITUTE OF ADVANCED INDUSTRIAL SCIENCE AND TECHNOLOGY, Tokyo (JP)

(72) Inventors: Shigehiro YOSHIDA, Saitama (JP); Hiroshi OIKAWA, Saitama (JP); Katsuya SHIMURA, Ibaraki (JP); Tadahiro FUJITANI, Ibaraki (JP)

(21) Appl. No.: 18/434,753

(22) Filed: Feb. 6, 2024

(30) Foreign Application Priority Data
Feb. 7, 2023 (JP) 2023-017135

(51) Int. Cl. C07C 5/27; C25B 3/03; C25B 3/26

(52) U.S. Cl. CPC C07C 5/2708; C25B 3/03; C25B 3/26; C07C 2529/06

(57) ABSTRACT

A method for generating isobutene, for isomerizing normal butene to isobutene, in which generation of by-products can be infinitely suppressed, and a high yield of isobutene can be achieved. The method for generating isobutene includes isomerizing normal butene to isobutene. In isomerizing, the normal butene is brought into contact with zeolite, and a reaction temperature in isomerizing is in a range from 25° C. to 249° C.

This application is based on and claims the benefit of priority from Japanese Patent Application No. 2023-017135, filed on 7 Feb. 2023, the content of which is incorporated herein by reference.

### BACKGROUND OF THE INVENTION

### Field of the Invention

The present invention relates to a method for generating isobutene, a catalyst for generating isobutene, and an isobutene generation system.

### Related Art

Conventionally, efforts to mitigate climate change or reduce its impact have been continuing. To achieve this, research and development on reduction of carbon dioxide is being carried out. For example, a technology is known which collects exhaust gas or carbon dioxide in the atmosphere and electrochemically reduces it to obtain a valuable substance. The above technology is a promising technology that can achieve carbon neutrality.

By electrochemically reducing carbon dioxide, ethylene is generated. When ethylene is subjected to oligomerization reaction in the presence of an oligomerization catalyst, olefin such as 1-butene is generated. Isobutene that is a structural isomer of normal butene including 1-butene is industrially important hydrocarbon and has various applications as a synthetic intermediate. For example, the addition reaction of isobutene with methanol or ethanol provides MTBE and ETBE used as additives for gasoline. Furthermore, dimerization and alkylation of isobutene provide isooctane to be added to gasoline. Therefore, technology on a catalyst for promoting the isomerization reaction of isomerizing normal butene to isobutene has been proposed (see, for example, Non-Patent Document 1).
Non-Patent Document 1: Journal of Catalysis 167, 273-278 (1997)

### SUMMARY OF THE INVENTION

Non-Patent Document 1 shows a result of obtaining a high yield of isobutene by isomerizing normal butene to isobutene in the presence of a zeolite catalyst such as a FER type zeolite. On the other hand, in the isomerization reaction disclosed in Non-Patent Document 1, by-products having 3 or 5 carbon atoms are generated in a considerable proportion. Therefore, it has been difficult to separate and collect unreactive normal butene after isomerization reaction. Furthermore, even if unreactive normal butene could be separated and collected, due to generation of by-products, the yield of isobutene was not able to have a predetermined yield or more. As mentioned above, in the method for isomerizing normal butene to isobutene, a technology to further improve the yield of isobutene has been demanded.

The present invention has been made in view of the foregoing, and an object of the present invention is to provide a method for generating isobutene that is a method for isomerizing normal butene to isobutene wherein generation of by-products can be infinitely suppressed and a high yield of isobutene can be achieved.

(1) The present invention relates to a method for generating isobutene, the method including isomerizing normal butene to isobutene, wherein in the isomerizing, the normal butene is brought into contact with zeolite, and a reaction temperature of the isomerizing is in a range from 25° C. to 249° C.

The invention of (1) can provide a method for generating isobutene by isomerizing normal butene to isobutene, in which generation of by-products can be infinitely suppressed, and a high yield of isobutene can be achieved.

(2) The method for generating isobutene described in (1), wherein a SiO₂/Al₂O₃ ratio of the zeolite is 2 to 1500.

The invention (2) can provide a method for generating isobutene in which generation of by-products can infinitely suppressed, and high isobutene yield is achieved.

(3) Furthermore, the present invention relates to a catalyst for generating isobutene for promoting an isomerization reaction of generating isobutene by isomerizing normal butene, wherein the catalyst for generating isobutene is zeolite, and a SiO₂/Al₂O₃ ratio of the zeolite is 2 to 1500.

The invention (3) can provide a catalyst for generating isobutene as a catalyst for promoting an isomerization reaction for generating isobutene by isomerizing normal butene to isobutene, wherein generation of by-products can be infinitely suppressed, and high yield of isobutene can be achieved.

(4) Furthermore, the present invention relates to an isobutene generation system including: an electrolyzer for generating ethylene by electrolyzing carbon dioxide;
a dimerizer for generating normal butene by dimerizing the ethylene; and
an isomerizer for generating isobutene by isomerizing the normal butene,
wherein the isomerizer includes a catalyst with which the normal butene is brought into contact, the catalyst is zeolite that is any one of FAU type, MFI type, BEA type, or MOR type, and
a SiO₂/Al₂O₃ ratio of the zeolite is 2 to 1500.

The invention of (4) can collect carbon dioxide in exhaust gas or the atmosphere and generate isobutene that is industrially important hydrocarbon, and therefore can contribute to achievement of carbon neutrality. Furthermore, in the isomerization reaction of generating isobutene by isomerizing normal butene to isobutene, generation of by-products can be infinitely suppressed and a high yield of isobutene can be provided.

(5) The isobutene generation system described in (4), further including a hydration reactor, wherein the hydration reactor separates the normal butene from a mixture including
the normal butene generated by the isomerizer and the isobutene, and
the normal butene separated by the hydration reactor is returned to the isomerizer.

According to the invention of (5), the yield of isobutene in the isomerizer of the isobutene generation system can be made to be a yield of theoretically 100%.

### BRIEF DESCRIPTION OF THE DRAWINGS

FIG. 1 is a block diagram showing a configuration of an isobutene generation system in accordance with an embodiment of the present invention; and

FIG. 2 is a graph showing a relationship between isobutene yield and flowing time in the method for generating isobutene in accordance with an embodiment.

FIG. 3 is a graph showing an isobutene yield for each type of catalyst.

### DETAILED DESCRIPTION OF THE INVENTION

### <Catalyst for Generating Isobutene>

A catalyst for generating isobutene in accordance with this embodiment is a catalyst for promoting isomerization reaction of isomerizing normal butene to isobutene. In this specification, the normal butene refers to a balanced mixture in which 1-butene, cis-2-butene, and trans-2-butene as structural isomers are present in a balanced state.

The catalyst for generating isobutene in accordance with this embodiment is zeolite. The zeolite is not particularly limited, and examples thereof include FAU type, MFI type, BEA type, MOR type, FER type, and CHA type zeolites. The FAU type, MFI type, BEA type, MOR type, FER type, and CHA type are codes defined by International Zeolite Association (IZA), and show skeletal structures of zeolite. For example, the maximum number of membered rings of the FAU type zeolite is 10. The intrinsic pore diameter of the FAU type zeolite is about 7.4 Å. Note here that since the molecular size of isobutene as the target substance is about 5.0 Å, it is considered that the intrinsic pore diameter of the FAU type zeolite is suitable for the isomerization reaction of isomerizing normal butene to isobutene.

The zeolite in accordance with this embodiment preferably has a SiO₂/Al₂O₃ ratio (material amount ratio) of 2 to 1500. When the zeolite having a SiO₂/Al₂O₃ ratio in the above range is used as a catalyst in the isomerization reaction of isomerizing normal butene to isobutene, the yield of isobutene can be improved. Furthermore, generation of by-products can be infinitely suppressed. Although the reason for the above is not certain, it is considered that the amount and distribution of acid sites on the zeolite surface become particularly suitable conditions for the isomerization reaction of isomerizing normal butene to isobutene by setting the SiO₂/Al₂O₃ ratio of the zeolite within the above range. From the viewpoint mentioned above, the SiO₂/Al₂O₃ ratio is preferably 500 to 1500. Furthermore, the zeolite in accordance with this embodiment is preferably Y type zeolite.

The zeolite as mentioned above may be produced by hydrothermal synthesis, or may be a commercial product (for example, HSZ-390HUA and the like manufactured by Tosoh Co., Ltd.).

The zeolite in accordance with this embodiment is preferably a proton type zeolite. The proton type zeolite can be obtained by well-known protonation treatment.

The acid strength of the zeolite in accordance with this embodiment is preferably less than 140E NH₃/kJmol⁻¹. This can suppress generation of by-products such as hydrocarbons having 3 or 5 carbon atoms, which are generated by cracking of hydrocarbon having 8 carbon atoms. Note here that the acid strength of zeolite in this specification is obtained by calculating the heat of adsorption of ammonia adsorbed at the acidic site of the zeolite by quantum chemical calculation called Density Functional Theory (DFT) calculation.

### <Method for Generating Isobutene>

A method for generating isobutene in accordance with this embodiment includes isomerizing in which normal butene as a raw material is brought into contact with the above zeolite that is a catalyst for promoting the isomerization reaction. The isomerization reaction of normal butene in the above isomerizing is represented by the following Formula (1).

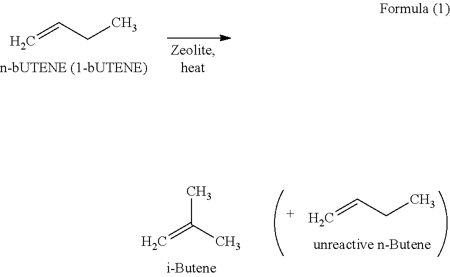

In the above Formula (1), normal butene (n-Butene) is described as 1-butene (1-Butene) for convenience, but the normal butene actually is a balanced mixture in which 1-butene, cis-2-butene, and trans-2-butene are present in a balanced state. The “i-Butene” in the above Formula (1) is isobutene. The “unreactive n-Butene” in the above Formula (1) is unreactive normal butene that is not isomerized by the isomerization reaction and is unreactive. In the above Formula (1), isobutene, and normal butene are described as products, but other than the above products, a small amount of by-products may be generated by the isomerization reaction shown by the above Formula (1).

In isomerizing, the reaction temperature is preferably in a range from 25° C. to 249° C. The reaction temperature is preferably in a range from 25° C. to 200° C., more preferably in a range from 25° C. to 150° C., and further preferably in a range from 25° C. to 100° C. Use of a catalyst for generating isobutene in accordance with this embodiment can not only lower the temperature of the isomerizing and reduce the cost required to the isomerizing, but also achieves a more preferable isobutene yield by lowering the reaction temperature.

In isomerizing, gas hourly space velocity (GHSV) of inlet gas including normal butene is preferably in a range from 20000 to 80000 ml·g⁻¹·h⁻¹. Furthermore, the contact time (W/F) is preferably in a range from 0.0008 to 0.0031 g·min·ml⁻¹. Thus, the conversion of normal butene can be improved. The conversion of the normal butene mentioned above is represented by the following Formula (2).

$$Normal butene conversion ( \% ) = ( ( Flow rate of normal butene of reaction gas ) - ( Material amount of normal butene at the inlet of the reactor ) ) / ( Flow rate of normal butene at the outlet of the reactor ) \times 100 \qquad ( 2 )$$

The selectivity of isobutene mentioned above is represented by the following Formula (3), and the yield of isobutene mentioned above is represented by the following Formula (4).

$$Isobutene selectivity ( \% ) = ( Amount of isobutene in generated gas ) / ( Total amount of products ) \times 100 \qquad ( 3 )$$

$$Isobutene yield ( \% ) = ( Isobutene selectivity ( \% ) ) \times ( Normal butene conversion ( \% ) ) \div 100 \qquad ( 4 )$$

The method for generating isobutene in accordance with this embodiment may include any steps other than the above isomerizing. For example, the method may include preparing reaction gas by diluting isobutene with N₂ gas at a predetermined diluting rate. Furthermore, the method may include separating unreactive normal butene from generated gas, and further include reusing the separated normal butene as a reaction gas. Since the method for generating isobutene in accordance with this embodiment can infinitely suppress the generation of by-products, unreactive normal butene can be easily separated from the generated gas. Furthermore, when the normal butene separated from the generated gas is used as a reaction gas, theoretical yield of isobutene can be improved to near 100%.

Separating of unreactive normal butene from the generated gas mentioned above can be achieved by, for example, a hydration reaction (water addition reaction) represented by the following Formula (5).

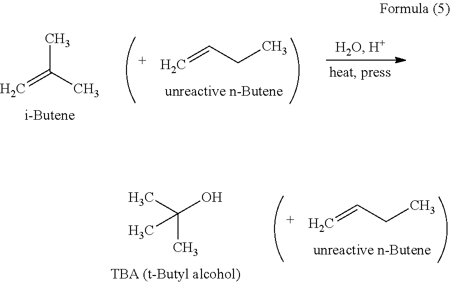

Since only isobutene can be converted to TBA (tert-butyl alcohol) that is a liquid or a solid at room temperature by the hydration reaction in the above Formula (5), normal butene that is gas at room temperature can be easily separated from the generated gas mentioned above. As a specific method, for example, a well-known method such as a method using an aqueous solution including a heteropolyacid having at least one element selected from Mo, W and V as a condensation coordination element and reacting at a temperature of less than 100° C. can be used. Note here that TBA can be effectively used by conversion to isooctane by well-known dimerization techniques, hydrogenation techniques, and the like. This is because isooctane can be used as a base material for gasoline.

### <Isobutene Generation System>

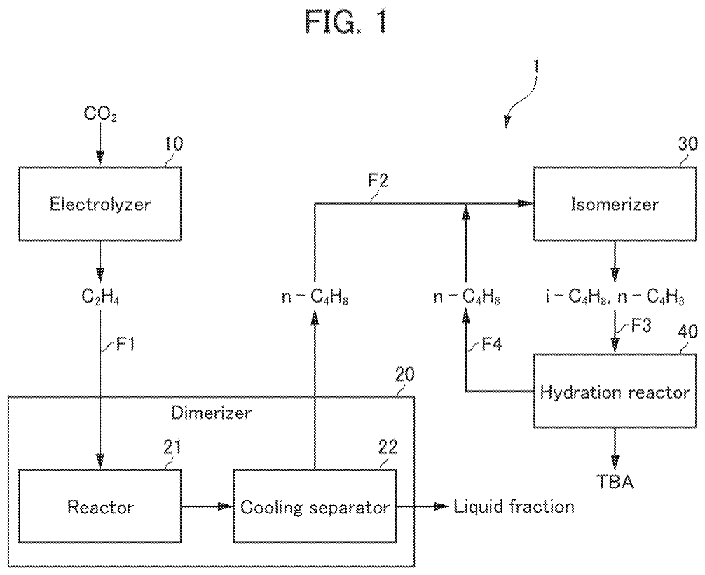

An isobutene generation system 1 in accordance with this embodiment includes, as shown in FIG. 1, an electrolyzer 10, a dimerizer 20, an isomerizer 30, a hydration reactor 40, and flow paths F1 to F4 for connecting them.

The electrolyzer 10 is a device for generating ethylene (C₂H₄) by electrochemically reducing carbon dioxide (CO₂). The electrolyzer 10 reduces carbon dioxide by an electrolytic cell for reducing carbon dioxide. Examples electrolytic cell include an electrolytic cell including at least a cathode and an anode. The cathode electrochemically reduces carbon dioxide to generate hydrocarbon such as ethylene (C₂H₄), and reduces water to generate hydrogen. The anode oxidizes hydroxide ions to generate oxygen. Ethylene (C₂H₄) generated by the electrolyzer 10 is supplied to the dimerizer 20 through the flow path F1.

A supply source of carbon dioxide (CO₂) supplied to the electrolyzer 10 is not particularly limited, and may be separated and collected from the air, or may be separated and collected from exhaust gas discharged from a combustion facility such as a boiler.

The dimerizer 20 is a device for dimerizing ethylene (C₂H₄) supplied through the flow path F1 by a dimerization reaction to generated normal butene (n-C₄H₈). The dimerizer 20 includes a reactor 21, and a cooling separator 22. The dimerizer 20 can generate normal butene (n-C₄H₈) in a yield of, for example, 80% or more.

The reactor 21 carries out an oligomerization reaction of ethylene in the presence of, for example, an olefin oligomerization catalyst to generate an olefin having the number of carbon atoms increased, such as normal butene (n-C₄H₈), 1-hexene or 1-octene. Examples of the olefin oligomerization catalyst include a solid acid catalyst using silica-alumina or zeolite as a carrier and a transition metal complex compound. Examples of metal atoms supported on the above carrier include Ni.

The cooling separator 22 carries out gas-liquid separation with respect to generated gas that has been subjected to an oligomerization reaction in the reactor 21. In the olefin having the number of carbon atoms increased included in the generated gas, since the boiling point rises in response to an increase in the carbon number, by setting the temperature of the cooling separator 22 to be not less than the boiling point of normal butene (n-C₄H₈) as the target substance and be less than the boiling point of the other olefins having 6 or more carbon atoms, normal butene (n-C₄H₈) and the other olefins having 6 or more carbon atoms can be easily gas-liquid separated. The normal butene (n-C₄H₈) separated by the cooling separator 22 is supplied to the isomerizer 30 through the flow path F2. The other olefins having 6 or more carbon atoms are separated and discharged as liquid fractions by the cooling separator 22.

The isomerizer 30 is a device for generating isobutene (i-C₄H₈) from normal butene (n-C₄H₈) supplied through the flow path F2. The isomerizer 30 includes the above zeolite. The zeolite is filled in, for example, a catalyst layer of a fixed bed reactor provided in the isomerizer 30. When gas including normal butene (n-C₄H₈) is allowed to flow through the above catalyst layer, normal butene (n-C₄H₈) and the zeolite are brought into contact with each other so that the isomerization reaction is promoted. The isomerizer 30 may include a diluter for diluting normal butene (n-C₄H₈) with N₂ gas at a predetermined diluting rate in addition to the above. Furthermore, well-known devices capable of adjusting the flow amount of the gas including normal butene (n-C₄H₈), as well as temperatures and pressure of the fixed bed reactor may be included. A mixture including isobutene (i-C₄H₈) generated by the isomerizer 30 and unreactive normal butene (n-C₄H₈) is supplied to the hydration reactor 40 through the flow path F3.

The hydration reactor 40 is a device for separating unreactive normal butene (n-C₄H₈) from the mixture including isobutene (i-C₄H₈) and unreactive normal butene (n-C₄H₈) supplied through the flow path F3 by the hydration reaction in the above Formula (5). Only isobutene (i-C₄H₈) is converted into TBA (tert-butyl alcohol) by a hydration reaction by the hydration reactor 40, and separated from the normal butene (n-C₄H₈) by gas-liquid separation. The TBA (tert-butyl alcohol) separated by the hydration reactor 40 is converted into isooctane and the like by the existing technique. The unreactive normal butene (n-C₄H₈) separated by the hydration reactor 40 is returned to the isomerizer 30 by the flow path F4 as the return flow path. The flow path F4 may be connected to the middle of the flow path F2 or may be connected to the isomerizer 30.

The isobutene generation system 1 having the above structure achieves the following effects. Since the isobutene generation system 1 has the isomerizer 30 having zeolite, the isobutene (i-C₄H₈) as the target substance can be obtained in a high yield, and generation of by-products can be infinitely suppressed. Furthermore, the isobutene generation system 1 includes a hydration reactor 40 for separating a mixture including isobutene (i-C₄H₈) generated by the isomerizer 30 and unreactive normal butene (n-C₄H₈), and the flow path F4 as the return flow path for returning unreactive normal butene (n-C₄H₈) separated by the hydration reactor 40 to the isomerizer 30. Thus, the yield of isobutene (i-C₄H₈) with respect to normal butene as raw material substance in the isomerizer 30 can be made be a yield of theoretically 100%.

Furthermore, the isobutene generation system 1 can generate isobutene that is an industrially important hydrocarbon by recovering carbon dioxide in exhaust gas and the atmosphere, and therefore contributes to achievement of carbon neutrality.

In the above, the preferable embodiments of the present invention are described, but the present invention is not limited to the above embodiments of the present invention, and modifications and improvements within a scope that can achieve the object of the present invention are included in the present invention.

In the above embodiments, the isobutene generation system 1 in which devices are directly connected through the flow paths F1 to F4 is described. Embodiments are not limited to the above. Each device may store a product in a storage tank such as a gas cylinder, and may supply a product to other devices by transporting the storage tank.

### Examples

Hereinafter, the present invention is described in detail with reference to Examples. However, the present invention is not limited to these Examples.

[Time Course of 1-Butene Conversion and is-Butene Yield]

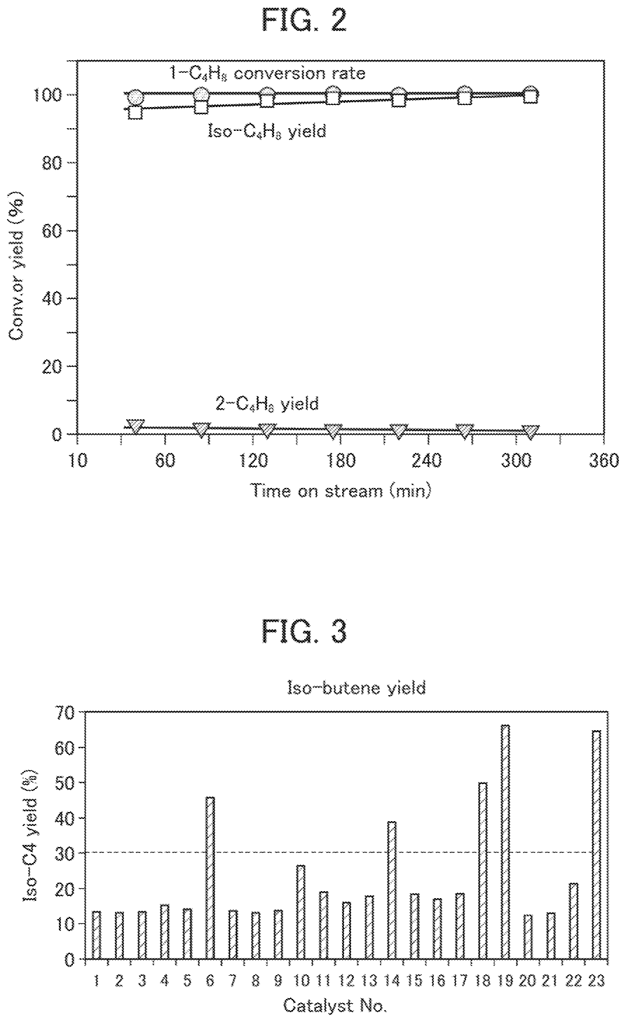

High silica H⁺—Y type zeolite (SiO₂/Al₂O₃=500, 390HUA, manufactured by Tosoh Co., Ltd.) was used, and the reaction gas is allowed to flow at a flow rate of normal butene (n-C₄H₈): 15 ml/min, N₂: 50 ml/min to cause an isomerization reaction. The reaction conditions include a temperature of 100° C., a pressure of 0.1 MPa, and a catalyst amount of 0.2 g. The outlet gas generated by the isomerization reaction was quantitatively analyzed by gas chromatography at different gas flow times (reaction times). Gas chromatography conditions are as follows. The results are shown in FIG. 2. In the graph of FIG. 2, the ordinate indicates the isobutene yield (%), and the abscissa indicates the gas flow time (reaction time) (min).

### (Measurement Conditions)

Measuring device: GC-2014 (manufactured by Shimadzu Corporation)
Columns: Rtx-1 (RESTEK, length: 60 m, inner diameter: 0.25 mm, thickness: 0.5 mm)
Carrier gas: N₂ (total flow rate: 50 ml/min, purge flow rate: 3.0 ml/min)
Split ratio: 66.1 (column flow rate: 0.70 ml/min) Injection: 250° C.

### Detection: 280° C.

Analysis: 10 min at 40° C., then increased to 250° C. at 20° C./min, and then 9.5 min at 250° C. (total 30 min)

As shown in FIG. 2, it is clear that when zeolite having a SiO₂/Al₂O₃ ratio of 500 is used and the reaction temperature is 100° C., a preferable isobutene yield can be obtained. Furthermore, it is clear that a yield of 2-C₄H₈ as by-products can be suppressed.

### [Relationship Between Reaction Temperature and Isobutene Yield]

The isomerization reaction of normal butene (n-C₄H₈) was carried out using each of catalysts shown in Tables 1 and 2 as catalysts at each reaction temperature. The reaction conditions are the same as in FIG. 2 except for the reaction temperature. The results are shown in Tables 1 and 2. Note here that in Tables 1 and 2, “yield/& iso-C₄H₈” indicates the isobutene yield.

TABLE 1

| | | Reaction | Conversion | | | Yield/% | | | | | | |
|---|---|---|---|---|---|---|---|---|---|---|---|---|
| | | temperature | rate/% | | | iso- | iso- | 2- | n- | | | C |
| Catalyst | | (° C.) | 1-C₄H₈ | C₂H₄ | C₃H₆ | C₄H₁₀ | C₄H₈ | C₄H₈ | C₄H₁₀ | C5 | C6 | balance |
| 760HOA | FER, 58 | 150 | 47.6 | 0 | 0 | 0.04 | 11.8 | 33.1 | 0 | 0 | 0 | 94.5 |
| | | 200 | 49.3 | 0 | 0.01 | 0.05 | 13.2 | 34.7 | 0 | 0 | 0 | 97.3 |
| | | 250 | 53.9 | 0.02 | 0.07 | 0.06 | 18.9 | 34.9 | 0 | 0.04 | 0 | 100 |
| | | 300 | 60.6 | 0.01 | 0.39 | 0.14 | 28.3 | 32 | 0 | 0.29 | 0.04 | 101 |
| | | 350 | 67.3 | 0.03 | 1.23 | 0.28 | 36 | 28.2 | 0.01 | 1 | 0.08 | 99.2 |
| 690HOA | MOR, 240 | 100 | 93.9 | 0 | 0 | 0.03 | 89.8 | 5.5 | 0 | 0 | 0 | 101.6 |
| | | 150 | 78.3 | 0.01 | 0 | 0.04 | 62.6 | 16.4 | 0 | 0 | 0 | 101 |
| | | 200 | 65.5 | 0 | 0.01 | 0.05 | 38.8 | 26.9 | 0 | 0 | 0.01 | 100.4 |
| | | 250 | 56.1 | 0.01 | 0.14 | 0.17 | 19.2 | 36.4 | 0 | 0.18 | 0.09 | 100.2 |
| | | 300 | 61.9 | 0.01 | 0.49 | 0.2 | 28.7 | 32.3 | 0 | 0.28 | 0.1 | 100.3 |
| | | 350 | 63 | 0.01 | 1.66 | 0.12 | 21 | 31.8 | 0.02 | 1.4 | 0.26 | 89.4 |
| 390HUA | Y, 500 | 65 | 100 | 0 | 0 | 0.04 | 98.6 | 0.9 | 0 | 0 | 0 | 99.6 |
| | | 100 | 99 | 0 | 0 | 0.04 | 94.7 | 2.7 | 0 | 0 | 0 | 98.4 |
| | | 150 | 87.4 | 0 | 0 | 0.04 | 68.3 | 19.7 | 0 | 0 | 0 | 100.8 |
| | | 200 | 78.9 | 0 | 0 | 0.04 | 49.7 | 29.7 | 0 | 0 | 0 | 100.6 |
| | | 250 | 72.3 | 0.04 | 0 | 0.04 | 39.1 | 31.8 | 0 | 0 | 0 | 98.2 |
| | | 300 | 55.8 | 0 | 0.02 | 0.04 | 19.6 | 36.3 | 0 | 0.07 | 0.03 | 100.6 |
| | | 350 | 59.6 | 0 | 0.24 | 0.07 | 22.7 | 34.5 | 0 | 0.51 | 0.17 | 97.6 |

TABLE 2

| | | Reaction | Conversion | | | Yield/% | | | | | | |
|---|---|---|---|---|---|---|---|---|---|---|---|---|
| | | temperature | rate/% | | | iso- | iso- | 2- | n- | | | C |
| Catalyst | | (° C.) | 1-C₄H₈ | C₂H₄ | C₃H₆ | C₄H₁₀ | C₄H₈ | C₄H₈ | C₄H₁₀ | C5 | C6 | balance |
| CBV28014 | ZSM-5, | 100 | 97.4 | 0 | 0 | 0.03 | 93.9 | 3.4 | 0 | 0 | 0 | 99.9 |
| | 280 | 150 | 77.3 | 0 | 0 | 0.04 | 56.8 | 21 | 0 | 0 | 0 | 100.8 |
| | | 200 | 54.8 | 0 | 0.02 | 0.03 | 21.4 | 33.2 | 0 | 0.05 | 0.05 | 100 |
| | | 250 | 78.6 | 0.02 | 3.32 | 0.18 | 11.9 | 16.1 | 0.13 | 10.43 | 6.08 | 61.3 |
| 890HOA | ZSM-5, | 100 | 98.4 | 0 | 0 | 0.03 | 96.9 | 2.2 | 0 | 0 | 0 | 100.7 |
| | 1500 | 150 | 94.1 | 0 | 0 | 0.03 | 88.4 | 6 | 0 | 0 | 0 | 100.4 |
| | | 200 | 81.5 | 0 | 0 | 0.04 | 64.6 | 16.6 | 0 | 0 | 0 | 99.7 |
| | | 250 | 61.3 | 0 | 0.98 | 0.04 | 14.4 | 30.7 | 0.01 | 2.27 | 1.8 | 81.8 |
| CP811E-150 | β, 150 | 100 | 93.1 | 0 | 0 | 0.04 | 83.2 | 11.5 | 0 | 0 | 0 | 101.8 |
| | | 150 | 91.5 | 0.01 | 0 | 0.03 | 79.6 | 12 | 0 | 0 | 0 | 100.1 |
| | | 200 | 75.5 | 0 | 0 | 0.04 | 45.8 | 30.4 | 0 | 0.01 | 0.01 | 101 |
| | | 250 | 55.4 | 0 | 0.12 | 0.09 | 16.6 | 35.3 | 0 | 0.57 | 0.3 | 95.7 |

Details of the catalysts of Tables 1 and 2 are as follows.
Ferrierite (manufactured by Tosoh Co., Ltd., 760HOA, FER (silica/alumina ratio: 58))
MOR type zeolite (manufactured by Tosoh Co., Ltd., 690HOA, MOR (silica/alumina ratio: 240))
FAU type (Y type) zeolite (manufactured by Tosoh Co., Ltd., 390HUA, Y (silica/alumina ratio: 500))
MFI type zeolite (manufactured by Zeolyst International, CBV28014, ZSM-5 (silica/alumina ratio: 280))
MFI type zeolite (manufactured by Tosoh Co., Ltd., 890HOA, ZSM-5 (silica/alumina ratio: 1500))
BEA type zeolite (manufactured by Zeolyst International, CP811E-150, β (silica/alumina ratio: 150))

As shown in Tables 1 and 2, results clearly shows that by carrying out an isomerization reaction of normal butene using zeolite, a high yield of isobutene is obtained. Furthermore, it is clear that when any one of zeolite of FAU type, MFI type, BEA type, and MOR type is used as types of zeolites, as the temperature becomes lower, in a temperature range of less than 250° C., higher yield of isobutene is obtained.

### [Relationship Between Type of Zeolite and Isobutene Yield]

Using catalysts shown in Table 3 below, reaction gas is allowed to flow at the flow rates of normal butene (n-C₄H₈) of 15 ml/min, and N₂ of 50 ml/min to cause the isomerization reaction. Reaction conditions include a temperature of 200° C., a pressure of 0.1 MPa, a reaction time of 40 min, and a catalyst amount of 0.2 g.

TABLE 3

| Cata- | | | | |
|---|---|---|---|---|
| lyst | | | Manufacture | SiO₂/Al₂O₃ |
| No. | Item number | Zeolite type | name | ratio |
| 1 | CP914C | FER | Zeolyst | 20 |
| | | | International | |
| 2 | 760HOA | FER | Tosoh Co., Ltd. | Around 60 |
| 3 | 770HOA | FER | Tosoh Co., Ltd. | 93 |
| 4 | CP814E | β (BEA) | Zeolyst | 25 |
| | | | International | |
| 5 | CP811E-75 | β (BEA) | Zeolyst | 75 |
| | | | International | |
| 6 | CP811E-150 | β (BEA) | Zeolyst | 150 |
| | | | International | |
| 7 | JRC-HB-150 | β (BEA) | Reference catalyst | 150 |
| | | | supplied from | |
| | | | Catalysis | |
| | | | Society of | |
| | | | Japan | |
| 8 | CP811C-300 | β (BEA) | Zeolyst | 300 |
| | | | International | |
| 9 | 980HOA | β (BEA) | Tosoh Co., Ltd. | 500 |
| 10 | 620HOA | MOR | Tosoh Co., Ltd. | 15 |
| 11 | JRC-Z-HM20 | MOR | Tosoh Co., Ltd. | 20 |
| 12 | 660HOA | MOR | Tosoh Co., Ltd. | 30 |
| 13 | JRC-Z-HM90 | MOR | Reference catalyst | 90 |
| | | | supplied from | |
| | | | Catalysis | |
| | | | Society of | |
| | | | Japan | |
| 14 | 690HOA | MOR | Tosoh Co., Ltd. | 240 |
| 15 | 350HUA | Y (FAU) | Tosoh Co., Ltd. | 10 |
| 16 | CBV901 | Y (FAU) | Zeolyst | 80 |
| | | | International | |
| 17 | 385HUA | Y (FAU) | Tosoh Co., Ltd. | 100 |
| 18 | 390HUA | Y (FAU) | Tosoh Co., Ltd. | 500 |
| 19 | 390HUA | Y (FAU) | Tosoh Co., Ltd. | 770 |
| 20 | CBV2314 | ZSM-5 (MFI) | Zeolyst | 23 |
| | | | International | |
| 21 | CBV8014 | ZSM-5 (MFI) | Zeolyst | 80 |
| | | | International | |
| 22 | CBV28014 | ZSM-5 (MFI) | Zeolyst | 280 |
| | | | International | |
| 23 | 890HOA | ZSM-5 (MFI) | Tosoh Co., Ltd. | 1500 |

### <Gas Chromatography>

Generated gas generated by the isomerization reaction with each catalyst shown in Table 3 was quantitatively analyzed by gas chromatography in the same conditions as in FIG. 2. Based on the analysis results obtained by the gas chromatography, the yield of isobutene is determined, and results are shown in the graph of FIG. 3.

### EXPLANATION OF REFERENCE NUMERALS

1 Isobutene generation system
10 Electrolyzer
20 Dimerizer
30 Isomerizer
40 Hydration reactor

What is claimed is:

1. A method for generating isobutene, the method comprising isomerizing normal butene to isobutene,
wherein in the isomerizing, the normal butene is brought into contact with zeolite, and
a reaction temperature of the isomerizing is in a range from 25° C. to 249° C.

2. The method for generating isobutene according to claim 1, wherein a SiO₂/Al₂O₃ ratio of the zeolite is 2 to 1500.

3. A catalyst for generating isobutene, promoting an isomerization reaction of isomerizing normal butene to generate isobutene,
wherein the catalyst for generating isobutene is zeolite, and
a SiO₂/Al₂O₃ ratio of the zeolite is 2 to 1500.

4. An isobutene generation system comprising:
an electrolyzer for generating ethylene by electrolyzing carbon dioxide;
a dimerizer for generating normal butene by dimerizing the ethylene; and
an isomerizer for generating isobutene by isomerizing the normal butene,
wherein the isomerizer includes a catalyst with which the normal butene is brought into contact,
the catalyst is zeolite, and
a SiO₂/Al₂O₃ ratio of the zeolite is 2 to 1500.

5. The isobutene generation system according to claim 4, further comprising a hydration reactor,
wherein the hydration reactor separates the normal butene from a mixture including the normal butene generated by the isomerizer and the isobutene, and
the normal butene separated by the hydration reactor is returned to the isomerizer.
